(19) United States
(12) Patent Application Publication
(10) Pub. No.: US 2010/0057219 A1
(43) Pub. Date: Mar. 4, 2010

(54) DEVICE SURFACE DESIGN FOR BETTER CELL ADHESION

(76) Inventor: Kwangyeol Lee, Namyangju-si (KR)

Correspondence Address: EDWARDS ANGELL PALMER & DODGE LLP, P.O. BOX 55874, BOSTON, MA, 02205, US

(21) Appl. No.: 12/201,855

(22) Filed: Aug. 29, 2008

(51) Int. Cl. A61F 2/02

(52) U.S. Cl. 623/23.76; 623/23.72

(57) ABSTRACT

Embodiments herein relate to devices promoting cell adhesion and methods of making thereof In some embodiments, one or more insoluble or non-degradable second surface components are positioned over one or more soluble or degradable first surface components over a surface. The first surface components can be degraded or dissolved to produce bumps or raised portions formed from the second surface components on the surface.

### BACKGROUND

Tissues or other biological structures may undergo damage or decay due to various circumstances, including disease, aging, or injury. Tissues that may incur damage include, for example, the heart, coronary vessels or artery, blood vessels, the spinal cord, bone, cartilage, tendon, ligament, breast, liver, gallbladder, bile duct, pancreas, intestinal, urinary system tissues, skin, and dental tissues.

One approach for treating tissue damage and/or defects includes implanting a medical device to facilitate tissue repair and generation. See, for example, U.S. Pat. Nos. 5,981,825 and 5,716,404, both of which are hereby incorporated by reference in their entireties. Another approach involves regenerating tissues from cells using ex vivo culture on a structural matrix and then introducing the regenerated tissues into a subject. Still another approach includes culturing cells and/or using extracellular matrix alone or in combination with artificial devices and subsequently implanting the cells in the body.

### SUMMARY

Some aspects of the disclosure relate to medical devices. The devices can include, for example, one or more first surface components that include one or more nanoparticles; and one or more second surface components that include one or more polymers. The second surface components can at least partly surround the first surface components.

The first component can be, for example, organic soluble or organic insoluble. The polymer can include, for example, a polyethylene glycol-based polymer. The polymer can be characterized by a molecular weight of at least about 10,000 g/mol or at least about 20,000 g/mol, for example. The second component can be, for example, hydrophilic. The nanoparticles can include, for example, a polymer. Also, in some aspects the adjacent nanoparticles can contact each other. The nanoparticles may be or include nanospheres or any other shape, for example. The second component can include a poly(lactic-co-glycolic-acid), for example. The second component can include, for example, poly(ester amide). The polymer can include, for example, polyethylene glycol-based polymer such as for example polyethylene glycol.

The coating further can include a biologically active agent, such as for example, antimicrobial agents, antibacterial agents, anti-fungal agents, anti-viral agents, anti-tumor agents, immunogenic agents, lipids, lipopolysaccharides, hormones, growth factors, and the like. The biologically active agent can include, for example, heparin, hirudin, hirulog, argatroban, D-phenylalanyl-L-poly-L-arginyl chloromethyl ketone, urokinase, streptokinase, aspirin, ticlopidine, colchicine, cytochalasin, methotrexate, dexamethasone, cyclosporin, trapidal, angiopeptin, dopamine, bromocriptine mesylate, pergolide mesylate, silver, captopril, enalapril, ascorbic acid, alphatocopherol, superoxide dismutase, deferoxyamine, a 21-aminosteroid (lasaroid), a thrombin inhibitor, an antithrombogenic agent, a tissue plasminogen activator, a thrombolytic agent, a fibrinolytic agent, a vasospasm inhibitor, a calcium channel blocker, a nitrate, a nitric oxide promoter, a vasodilator, an antimicrobial agent, an antibiotic agent, a glycoprotein IIb/IIIa inhibitor, an inhibitor of surface glycoprotein receptors, an antiplatelet agent, an antimitotic agent, a microtubule inhibitor agent, a retinoid, an antisecretory agent, an actin inhibitor, a remodeling inhibitor, a deoxyribonucleic acid, an antisense nucleotide, an antiproliferative agent, an anti-cancer chemotherapeutic agent, an anti-inflammatory steroid, a non-steroidal anti-inflammatory agent, an immunosuppressive agent, a PDGF antagonist agent, a growth hormone antagonist agent, an anti-growth factor antibody, a growth factor, a growth factor antagonist, a dopamine agonist agent, a radiotherapeutic agent, a radiopaque agent, a peptide, a protein, an enzyme, an extracellular matrix component, a second angiotensin converting enzyme (ACE) inhibitor, a free radical scavenger agent, an iron chelator agent, an antioxidant agent, and a radiolabeled agent.

The medical device be, for example, catheters, fibers, non-woven fabrics, vascular grafts, porous metals for acetabulum revision, cardiac pacemakers, tachyarrhythmia control devices, dental filling materials, materials used in osteo-synthesis, cardiac patches, sutures, soft and hard tissue scaffolds and fillers, stents, bone void fillers intended for the repair of bone defects, intrauterine devices, root canal fillers, drug delivery pumps, implantable infusion pumps, spacer devices, implants containing medicinal products, scaffolds for tissue engineering, and the like.

Some aspects of the disclosure relate to methods of treating a patient, the methods including, for example, at least partially removing the one or more first components of a device as described above and elsewhere herein; and implanting the device in the patient.

Also, some aspects of the disclosure relate to surface layers or substrates that include, for example, one or more first surface components that include one or more nanoparticles; and one or more second surface components including one or more polymers, the second surface components at least partly surrounding the first surface components, wherein the surface layer is configured for attachment, for example, to a surface of an implantable medical device. In some aspects the first one or more first surface components and/or the one or more second surface components can be organic soluble or insoluble, for example. The layer can include a film, for example. Also, the layer can include an adhesive component. In some aspects the layers can be utilized in methods of treating a patient. For example, such methods can include attaching a layer as described above or elsewhere herein to the surface of the implantable medical device; and implanting the device in the patient. The methods can be used to treat, for example, a patient suffering from stenosis, a heart condition, a dental condition, a vascular condition, cancer, incontinence or the like.

Some aspects of the disclosure relate to methods of manufacturing an implantable medical device. The methods can include attaching a layer as described herein to the surface of the implantable medical device. Some aspects relate to devices formed by the methods described herein. The layers described above or elsewhere herein can be used in methods of treating a patient, which methods can include attaching a layer described above or elsewhere herein to the surface of the implantable medical device; and implanting the device in the patient. The patient can be, for example, suffering from stenosis, a heart condition, a dental condition, a vascular condition, cancer, incontinence, and the like.

Further aspects of the disclosure relate to methods of manufacturing an implantable medical device. The methods can include, for example, at least partially contacting the medical device with one or more first surface components including one or more nanoparticles; and at least partially contacting the medical device with one or more second surface components including one or more polymers, such that the second surface components at least partly surround the first surface components. Some aspects relate to devices formed by the methods described herein.

The one or more first surface components can be, for example, organic soluble. The one or more second surface components can be, for example, organic insoluble. The polymer can include, for example, a polyethylene glycol-based polymer. The at least partially contacting the medical device with the one or more first surface components can include at least partially coating the medical device with the one or more first surface components and/or one or more second surface components, for example. The at least partially contacting the medical device with the one or more first surface components can include, for example, one or more of brushing, spraying, wiping, dipping, extruding and/or injecting the medical device with the first component, or any other suitable process. The at least partially contacting the medical device with the one or more second surface components can include, for example, brushing, spraying, wiping, dipping, extruding and/or injecting the medical device with the second component, or any other suitable process. The methods further can include preparing a coating solution including the first and second components, wherein at least partially contacting the medical device with the one or more first surface components and at least partially contacting the medical device with the one or more second surface components includes at least partially contacting the medical device with the coating solution. The coating solution further can include a biologically active agent, for example. In some aspects the polymer can be characterized, for example, by a molecular weight of at least about 10,000 g/mol, at least about 20,000 g/mol, or the like. The one or more first surface components can be, for example, hydrophilic. The nanoparticles can include, for example, a polymer. The adjacent nanoparticles can contact each other or they may not be in contact. The nanoparticles can include nanospheres, for example. The methods further can include contacting at least part of the medical device with an organic solvent. The contacting the medical device with an organic solvent can detach at least part of the one or more first surface components from the medical device. The medical devices can be, for example, catheters, fibers, non-woven fabrics, vascular grafts, porous metals for acetabulum revision, cardiac pacemakers, tachyarrhythmia control devices, dental filling materials, materials used in osteo-synthesis, cardiac patches, sutures, soft and hard tissue scaffolds and fillers, stents, bone void fillers intended for the repair of bone defects, intrauterine devices, root canal fillers, drug delivery pumps, implantable infusion pumps, spacer devices, implants containing medicinal products, scaffolds for tissue engineering or the like.

Some aspects of the disclosure relate to methods of manufacturing a layer configured for attachment to a surface of an implantable medical device. The methods can include, for example, at least partially contacting a medical device with one or more first surface components including one or more nanoparticles; and at least partially contacting the medical device with one or more second surface components including one or more polymers, such that the second surface components at least partly surrounding the first surface components. In some aspects, the layer can include an adhesive component, which, for example, can be used to attach the layer to the medical device. The first component can be, for example, organic soluble. The second component can be, for example, organic insoluble. The methods further can include at least partially removing the one or more first components.

Some aspects of the disclosure relate to medical devices, including implantable devices, that include one or more first components including one or more biodegradable polymers at least partially covering one or more surfaces of the medical device; and one or more second components including one or more non-biodegradable polymers at least partially positioned over the first surface layer. Some aspects relate to implantable medical devices that include one or more non-biodegradable polymers at least partially coating one or more surfaces of the medical device, wherein the one or more non-biodegradable polymers are characterized by one or more raised portions. Still further aspects of the disclosure relate to methods of treating a patient. The methods can include, for example, implanting a device described herein into a patient.

The one or more first components and/or the one or more second components can be or can include a strip, for example. In some aspects, the one or more first components can include at least one strip, and the one or more second components can include at least one strip. The one or more first components and/or the one or more second components can be or can include a fiber, for example. The first and second components can be configured, for example, such that a plurality of cavities is formed between the one or more second components and the surface upon degradation of the one or more first components. The dimensions (e.g., height, width, diameter, length, etc.) of the strips, fibers or other surface component can be of any suitable size for the particular application and/or for the size of the surface of the device or the layer, for example. The strips, fibers or other first or second components can include one or more dimensions, such as a height, a width, a diameter, etc. of between about 1 nm and 1000 nm. In some instances, one or more dimensions of the strip or fiber can be less than about, or about 1000, 500, 300, 100, 50, 30, 10, 5, 3, or 1 nm, for example. The adjacent cavities can be, for example, separated by a distance greater than about 50 nm and less than about 300 nm. The first component strips can be positioned substantially perpendicular to the second component strips, for example. The strips can be positioned to form a plurality of intersections between the biodegradable polymer strip and the non-biodegradable polymer strip, for example. The strips are positioned such that the biodegradable polymer strip raises portions of the non-biodegradable polymer strip. The one or more second components can include, for example, a polyethylene glycol-based polymer. The one or more second components can include a polymer with a molecular weight of at least about 10,000 g/mol and/or at least about 20,000 g/mol, for example. The one or more second components can include, for example, a polymer such as aliphatic polyesters; poly(glycolic acid) and/or copolymers thereof (e.g., poly(glycolide trimethylene carbonate); poly(caprolactone glycolide)); poly(lactic acid) and/or isomers thereof (e.g., poly-L(lactic acid) and/or poly-D Lactic acid) and/or copolymers thereof (e.g. DL-PLA), with and without additives (e.g. calcium phosphate glass), and/or other copolymers (e.g., poly(caprolactone lactide), poly(lactide glycolide), poly(lactic acid ethylene glycol)); poly(ethylene glycol); poly(ethylene glycol) diacrylate; poly(lactide); polyalkylene succinate; polybutylene diglycolate; polyhydroxybutyrate (PHB); polyhydroxyvalerate (PHV); polyhydroxybutyrate/polyhydroxyvalerate copolymer (PHB/PHV); poly(hydroxybutyrate-covalerate); polyhydroxyalkaoates (PHA); polycaprolactone; poly(caprolactone-polyethylene glycol) copolymer; poly(valerolactone); polyanhydrides; poly(orthoesters) and/or blends with polyanhydrides; poly(anhydride-co-imide); polycarbonates (aliphatic); poly(hydroxyl-esters); polydioxanone; polyanhydrides; polyanhydride esters; polycyanoacrylates; poly(alkyl 2-cyanoacrylates); poly(amino acids); poly(phosphazenes); poly(propylene fumarate); poly(propylene fumarate-co-ethylene glycol); poly(fumarate anhydrides); fibrinogen; fibrin; gelatin; cellulose and/or cellulose derivatives and/or cellulosic polymers (e.g., cellulose acetate, cellulose acetate butyrate, cellulose butyrate, cellulose ethers, cellulose nitrate, cellulose propionate, cellophane); chitosan and/or chitosan derivatives (e.g., chitosan NOCC, chitosan NOOC-G); alginate; polysaccharides; starch; amylase; collagen; polycarboxylic acids; poly(ethylester-co-carboxylate carbonate) (and/or other tyrosine derived polycarbonates); poly(iminocarbonate); poly(BPA-iminocarbonate); poly(trimethylene carbonate); poly(iminocarbonate-amide) copolymers and/or other pseudo-poly(amino acids); poly(ethylene glycol); poly(ethylene oxide); poly(ethylene oxide)/poly(butylene terephthalate) copolymer; poly(epsilon-caprolactone-dimethyltrimethylene carbonate); poly(ester amide); poly(amino acids) and conventional synthetic polymers thereof; poly(alkylene oxalates); poly(alkylcarbonate); poly(adipic anhydride); nylon copolyamides; NO-carboxymethyl chitosan NOCC); carboxymethyl cellulose; copoly(ether-esters) (e.g., PEO/PLA dextrans); polyketals; biodegradable polyethers; biodegradable polyesters; polydihydropyrans; polydepsipeptides; polyarylates (L-tyrosine-derived) and/or free acid polyarylates; polyamides (e.g., Nylon 66, polycaprolactam); poly(propylene fumarate-co-ethylene glycol) (e.g., fumarate anhydrides); hyaluronates; poly-p-dioxanone; polypeptides and proteins; polyphosphoester; polyphosphoester urethane; polysaccharides; pseudo-poly(amino acids); starch; terpolymer; (copolymers of glycolide, lactide, or dimethyltrimethylene carbonate); rayon; rayon triacetate; latex; and/copolymers, blends, and/or composites of above. The medical device can be, for example, one or more of catheters, fibers, non-woven fabrics, vascular grafts, porous metals for acetabulum revision, cardiac pacemakers, tachyarrhythmia control devices, dental filling materials, materials used in osteo-synthesis, cardiac patches, sutures, soft and hard tissue scaffolds and fillers, stents, bone void fillers intended for the repair of bone defects, intrauterine devices, root canal fillers, drug delivery pumps, implantable infusion pumps, spacer devices, implants containing medicinal products, scaffolds for tissue engineering, and the like.

Also, some aspects of the disclosure relate to methods of treating a patient. The methods can include, for example, at least partially removing the one or more second components of a device as described herein; and implanting the device in the patient. The patient may be, for example, one suffering from stenosis, a heart condition, a dental condition, a vascular condition, cancer, incontinence and the like. The treatment can be such that cells of the patient anchor to the one or more second components of the medical device following implantation of the device.

Further aspects of the disclosure relate to layers configured for attachment to a surface of an implantable medical device. The layers can include, for example, a biodegradable polymer strip over a substrate; and one or more non-biodegradable polymers over the biodegradable polymer strip. Some aspects of the disclosure relate to layers configured for attachment to a surface of an implantable medical device. The layers can include, for example, one or more non-biodegradable polymers at least partially coating a substrate, wherein the one or more non-biodegradable polymers are characterized by one or more raised portions. The biodegradable polymer strip can include, for example, a biodegradable polymer fiber. The one or more non-biodegradable polymers can include, for example, one or more of a strip, a fiber, and the like. The strips can be configured such that a plurality of cavities is formed between the non-biodegradable polymer strip and the surface upon degradation of the biodegradable polymer. The strips can be positioned such that the biodegradable polymer strip raises portions of the non-biodegradable polymer strip. The layer can include, for example, a film. Also, the layer can include, for example, an adhesive component.

Some embodiments relate to methods of manufacturing an implantable medical device. The methods can include, for example, attaching the layer described herein to the surface of the implantable medical device. Some aspects relate to devices formed by the method described herein.

Some aspects relate to methods of treating a patient, which methods can include for example, attaching a layer as described herein to the surface of an implantable medical device; and implanting the device in the patient.

Further aspects relate to methods of manufacturing an implantable medical device, which methods can include, for example, positioning one or more first components at least partially over one or more surfaces of the medical device, the one or more first components including one or more biodegradable polymers; and positioning one or more second components at least partially over the one or more first components, the one or more second components including one or more non-biodegradable polymers. Some aspects relate to devices formed by the method described herein.

The one or more first components and/or the one or more second components can include, for example, one or more of a strip and a fiber. The first component strips can be positioned, for example, substantially perpendicular to the second component strips. The strips can be positioned, for example, to form a plurality of intersections between the biodegradable polymer strip and the non-biodegradable polymer strip. The strips can be positioned, for example, such that the first component strip raises portions of the second component strip. The dimensions (e.g., height, width, diameter, length, etc.) of the strips, fibers or other surface component can be of any suitable size for the particular application and/or for the size of the surface of the device or the layer, for example. The strips, fibers or other first or second components can include one or more dimensions, such as a height, a width, a diameter, etc. of between about 1 nm and 1000 nm. In some instances, one or more dimensions of the strip or fiber can be less than about, or about 1000, 500, 300, 100, 50, 30, 10, 5, 3, or 1 nm, for example. The one or more second components can include, for example, a polyethylene glycol-based polymer. The methods further can include, for example, removing the one or more first components. The removing can include, for example, contacting the one or more first components with a solvent. The removing can result in the formation of a plurality of cavities between the one or more second components and the surface. The one or more second components can include, for example, a polymer with a molecular weight of at least about 10,000 g/mol and/or at least about 20,000 g/mol, for example. The one or more second components can include, for example one or more of the following polymers aliphatic polyesters; poly(glycolic acid) and/or copolymers thereof (e.g., poly(glycolide trimethylene carbonate); poly(caprolactone glycolide)); poly(lactic acid) and/or isomers thereof (e.g., poly-L(lactic acid) and/or poly-D Lactic acid) and/or copolymers thereof (e.g. DL-PLA), with and without additives (e.g. calcium phosphate glass), and/or other copolymers (e.g., poly(caprolactone lactide), poly(lactide glycolide), poly(lactic acid ethylene glycol)); poly(ethylene glycol); poly(ethylene glycol) diacrylate; poly(lactide); polyalkylene succinate; polybutylene diglycolate; polyhydroxybutyrate (PHB); polyhydroxyvalerate (PHV); polyhydroxybutyrate/polyhydroxyvalerate copolymer (PHB/PHV); poly(hydroxybutyrate-covalerate); polyhydroxyalkaoates (PHA); polycaprolactone; poly(caprolactone-polyethylene glycol) copolymer; poly(valerolactone); polyanhydrides; poly(orthoesters) and/or blends with polyanhydrides; poly(anhydride-co-imide); polycarbonates (aliphatic); poly(hydroxyl-esters); polydioxanone; polyanhydrides; polyanhydride esters; polycyanoacrylates; poly(alkyl 2-cyanoacrylates); poly(amino acids); poly(phosphazenes); poly(propylene fumarate); poly(propylene fumarate-co-ethylene glycol); poly(fumarate anhydrides); fibrinogen; fibrin; gelatin; cellulose and/or cellulose derivatives and/or cellulosic polymers (e.g., cellulose acetate, cellulose acetate butyrate, cellulose butyrate, cellulose ethers, cellulose nitrate, cellulose propionate, cellophane); chitosan and/or chitosan derivatives (e.g., chitosan NOCC, chitosan NOOC-G); alginate; polysaccharides; starch; amylase; collagen; polycarboxylic acids; poly(ethylester-co-carboxylate carbonate) (and/or other tyrosine derived polycarbonates); poly(iminocarbonate); poly(BPA-iminocarbonate); poly(trimethylene carbonate); poly(iminocarbonate-amide) copolymers and/or other pseudo-poly(amino acids); poly(ethylene glycol); poly(ethylene oxide); poly(ethylene oxide)/poly(butylene terephthalate) copolymer; poly(epsilon-caprolactone-dimethyltrimethylene carbonate); poly(ester amide); poly(amino acids) and conventional synthetic polymers thereof; poly(alkylene oxalates); poly(alkylcarbonate); poly(adipic anhydride); nylon copolyamides; NO-carboxymethyl chitosan NOCC); carboxymethyl cellulose; copoly(ether-esters) (e.g., PEO/PLA dextrans); polyketals; biodegradable polyethers; biodegradable polyesters; polydihydropyrans; polydepsipeptides; polyarylates (L-tyrosine-derived) and/or free acid polyarylates; polyamides (e.g., Nylon 66, polycaprolactam); poly(propylene fumarate-co-ethylene glycol) (e.g., fumarate anhydrides); hyaluronates; poly-p-dioxanone; polypeptides and proteins; polyphosphoester; polyphosphoester urethane; polysaccharides; pseudo-poly(amino acids); starch; terpolymer; (copolymers of glycolide, lactide, or dimethyltrimethylene carbonate); rayon; rayon triacetate; latex; and/copolymers, blends, and/or composites of above. The medical device can be or include, for example, catheters, fibers, non-woven fabrics, vascular grafts, porous metals for acetabulum revision, cardiac pacemakers, tachyarrhythmia control devices, dental filling materials, materials used in osteo-synthesis, cardiac patches, sutures, soft and hard tissue scaffolds and fillers, stents, bone void fillers intended for the repair of bone defects, intrauterine devices, root canal fillers, drug delivery pumps, implantable infusion pumps, spacer devices, implants containing medicinal products, scaffolds for tissue engineering, and the like. Some aspects relate to devices formed by the methods described above or elsewhere herein.

Also, some aspects relate to methods of manufacturing a layer configured for attachment to a surface of an implantable medical device. The methods can include, for example, positioning a biodegradable polymer strip at least partially over a substrate; and positioning one or more non-biodegradable polymers at least partially over the biodegradable polymer strip. The dimensions (e.g., height, width, diameter, length, etc.) of the strips or other surface component can be of any suitable size for the particular application and/or for the size of the surface of the device or the layer, for example. The strips can include one or more dimensions, such as a height, a width, a diameter, etc. of between about 1 nm and 1000 nm, for example. In some instances, one or more dimensions of the strips can be less than about, or about 1000, 500, 300, 100, 50, 30, 10, 5, 3, or 1 nm, for example. The layer can include, for example, an adhesive component. The biodegradable polymer strip can include, for example, a fiber. The one or more non-biodegradable polymers can include, for example, one or more of a strip, a fiber and the like. The strips can be positioned, for example, such that the biodegradable polymer strip raises portions of the one or more non-biodegradable polymers. The methods further can include removing the biodegradable polymer strip.

Some aspects of the disclosure relate to implantable medical devices that include, for example, one or more non-biodegradable polymers at least partially coating one or more surfaces of the medical device, wherein the one or more non-biodegradable polymers are characterized by one or more raised portions. The one or more non-biodegradable polymers can include, for example, at least one strip and/or at least one fiber. The raised portions can be, for example, substantially evenly spaced. The adjacent raised portions can be, for example, separated by a distance greater than about 50 nm and less than about 300 nm. The raised portions can be, for example, approximately one strip thickness above non-raised portions of the strip. The one or more non-biodegradable polymers can include, for example, a polyethylene glycol-based polymer. The one or more non-biodegradable polymers can include, for example, a polymer with a molecular weight of at least about 10,000 g/mol and/or at least about 20,000 g/mol. The device can be or include one or more of catheters, fibers, non-woven fabrics, vascular grafts, porous metals for acetabulum revision, cardiac pacemakers, tachyarrhythmia control devices, dental filling materials, materials used in osteo-synthesis, cardiac patches, sutures, soft and hard tissue scaffolds and fillers, stents, bone void fillers intended for the repair of bone defects, intrauterine devices, root canal fillers, drug delivery pumps, implantable infusion pumps, spacer devices, implants containing medicinal products, scaffolds for tissue engineering, and the like.

Some aspects relate to methods of treating a patient, which methods can include, for example, implanting a device as described above or elsewhere herein into a patient.

Also, some aspects of the disclosure relate to layers configured for attachment to a surface of an implantable medical device. The layers can include, for example, one or more non-biodegradable polymers at least partially coating a substrate, wherein the one or more non-biodegradable polymers are characterized by one or more raised portions. The layer can include, for example, a film. The layer can include, for example, an adhesive component. The one or more non-biodegradable polymers can include, for example, at least one of a strip, a fiber, and the like. The layers can be utilized in methods of treating a patient, for example, which can include attaching a layer as described above or elsewhere herein to the surface of an implantable medical device; and implanting the device in a patient.

The foregoing is a summary and thus contains, by necessity, simplifications, generalization, and omissions of detail; consequently, those skilled in the art will appreciate that the summary is illustrative only and is not intended to be in any way limiting. Other aspects, features, and advantages of the devices and/or processes and/or other subject matter described herein will become apparent in the teachings set forth herein.

### BRIEF DESCRIPTION OF THE DRAWINGS

The foregoing and other features of the present disclosure will become more fully apparent from the following description and appended claims, taken in conjunction with the accompanying drawings. Understanding that these drawings depict only several embodiments in accordance with the disclosure and are not to be considered limiting of its scope, the disclosure will be described with additional specificity and detail through use of the accompanying drawings.

FIGS. 1A-1B show illustrative surfaces for promoting cell adhesion formed by one or more first surface components that include nanoparticles and one or more second particles at least partially surrounding the one or more first surface components.

FIGS. 2A-2E show illustrative surfaces for promoting cell adhesion formed by one or more first surface components that include nanoparticles and one or more second particles at least partially surrounding the one or more first surface components.

FIGS. 3A-3D show an illustrative top-down view of a surface that includes protrusions or elevated features.

FIG. 4 describes an illustrative process of making a device with a cell-adhesion promoting surface.

### DETAILED DESCRIPTION

In the following detailed description, reference is made to the accompanying drawings, which form a part hereof In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented here. It will be readily understood that the aspects of the present disclosure, as generally described herein, and illustrated in the Figures, can be arranged, substituted, combined, and designed in a wide variety of different configurations, all of which are explicitly contemplated and make part of this disclosure.

For certain devices it may desirable for cells to adhere to the devices. For example, cell adhesion can be desirable for implanted medical devices. Such adherence of cells can allow for the device introduced into the body to be successfully incorporated into the body or to be successfully integrated in or near a regenerated tissue. Also, cells that adhere to the device may then differentiate into tissue forming cells.

Embodiments herein relate to compositions of matter and devices that can be used to promote or improve cell adhesion to a surface. Some embodiments relate to methods of making the compositions of matter and devices. In some aspects the devices can include features, such as protrusions or elevations, to which cells or tissue can adhere. The features (e.g., elevations or protrusions) can provide increased surface area to which cells or tissue can more effectively attach.

In some embodiments, one or more insoluble or non-degradable components can be positioned around and/or over one or more soluble or degradable components on a surface. The soluble/degradable components can be removed (e.g., degraded or dissolved) to produce features, such as protrusions or raised portions) that are formed from the insoluble or non-degradable components on the surface.

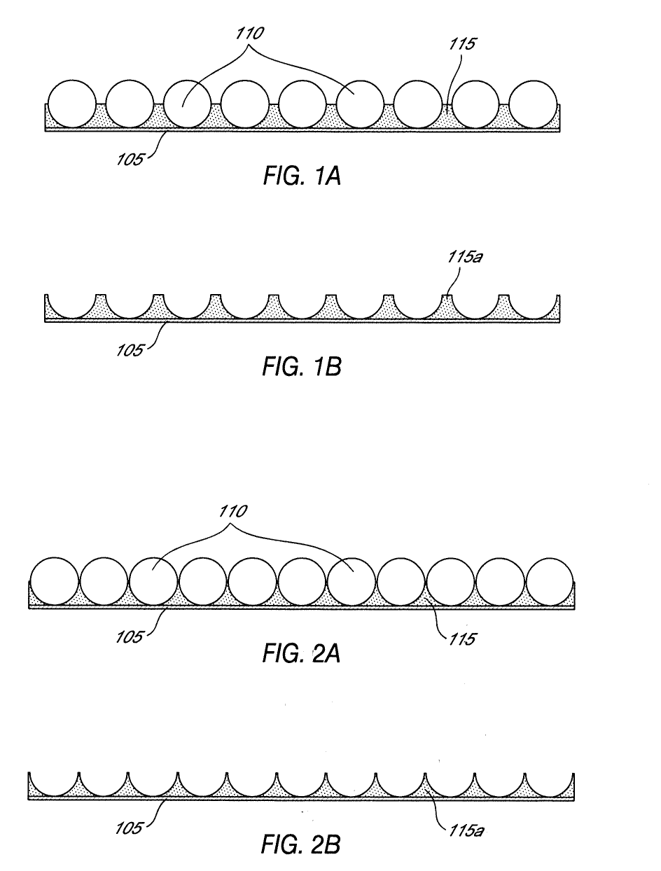

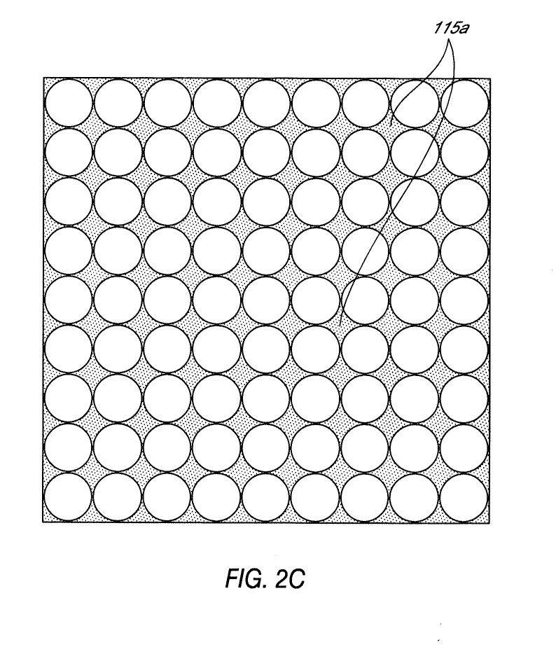

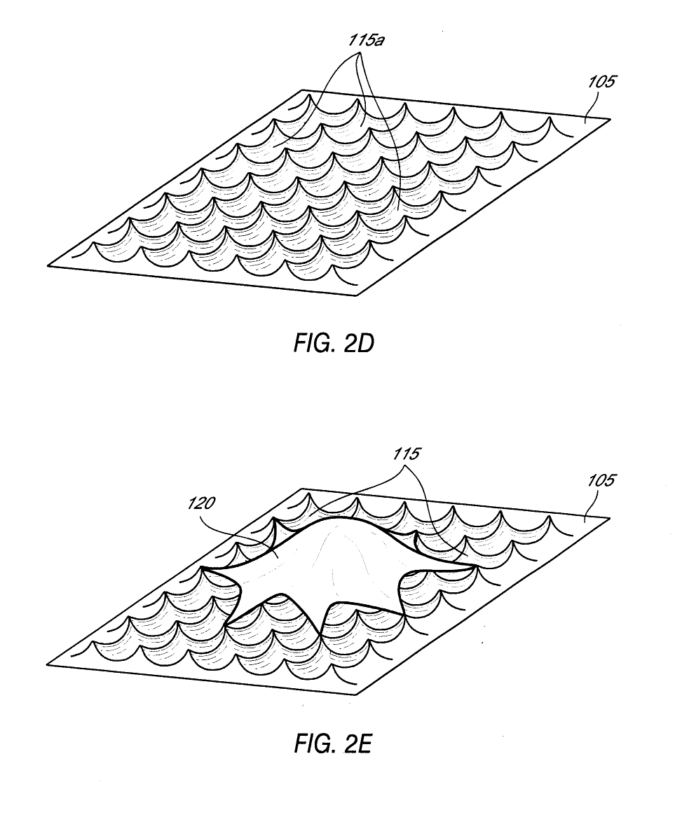

Illustrative and non-limiting examples of devices according to some aspects of the disclosure are depicted in FIGS. 1-3. FIGS. 1A, 1B, and 2A-2D show surfaces (e.g., a substrate surface or medical device surface) that can promote improved cell adhesion. One or more first surface components can be positioned on the substrate 105. For example, the one or more first surface components may include, but are not limited to, one or more nanoparticles 110 that are degradable and/or soluble. For example, the nanoparticles 110 can be biodegradable and/or soluble in an organic solvent. Further, one or more second surface components 115 can be deposited onto the substrate 105 on and/or around the nanoparticles 110. For example, the one or more second surface components 115 can at least partially surround or “fill in” the space on the surface of substrate that has not been filled in by the nanoparticles 115. FIGS. 1A and 2A illustrate two examples where the second surface component 115 at least partially surrounds or fills in around the nanoparticles 115. The one or more second surface components may include but is not limited to, for example, one or more polymers, including for example, non-degradable or insoluble polymers. Additional details of illustrative processes for making the surfaces illustrated in FIGS. 1A, 1B and 2A-2D are provided below.

FIG. 1A illustrates an example of a configuration where the nanoparticles 110 are separated from each other with at least part of the intervening space occupied by the second surface component 115. FIG. 2A illustrates an example of a configuration where adjacent nanoparticles 115 are at least partially touching or have very little space in between adjacent nanoparticles.

The one or more first surface components, for example, nanoparticles can be at least partially removed from the substrate. The removal can be accomplished, for example, by degrading and/or dissolving the nanoparticles. The nanoparticles 110 can be removed for example using an organic solvent, water, or some other degradative process, which process will not degrade the second surface components 115 (at least not at the same rate (e.g., significantly or substantially less rapidly) as it degrades the first component/nanoparticle). FIGS. 1B and 2B depict illustrative surfaces where the nanoparticles 110 have been partially or completely removed, thus, leaving the substrate 105 with the one or more second surface components 115a. The one or more second surface components 115a may thus form elevated features such as protrusions or peaks on the substrate 105. The elevated features 115a may be formed on the substrate 105 in pattern. For example the pattern can be a regular pattern or an at least semi-regular pattern (e.g., where spacing between adjacent bumps is substantially similar across bumps), as well as random or irregular patterns.

FIG. 2C depicts a top view of an illustrative surface with the nanoparticles 110 removed thereby leaving a pattern of elevated features 115a on the substrate 105. The elevated features were formed in the various locations between adjacent nanoparticles.

FIG. 2D depicts an illustrative plain view of the substrate 105 with the elevated features made up of the second surface component 115a that remained after removing the first surface component 110. Furthermore, FIG. 2E illustrates a cell 120 attached to the surface.

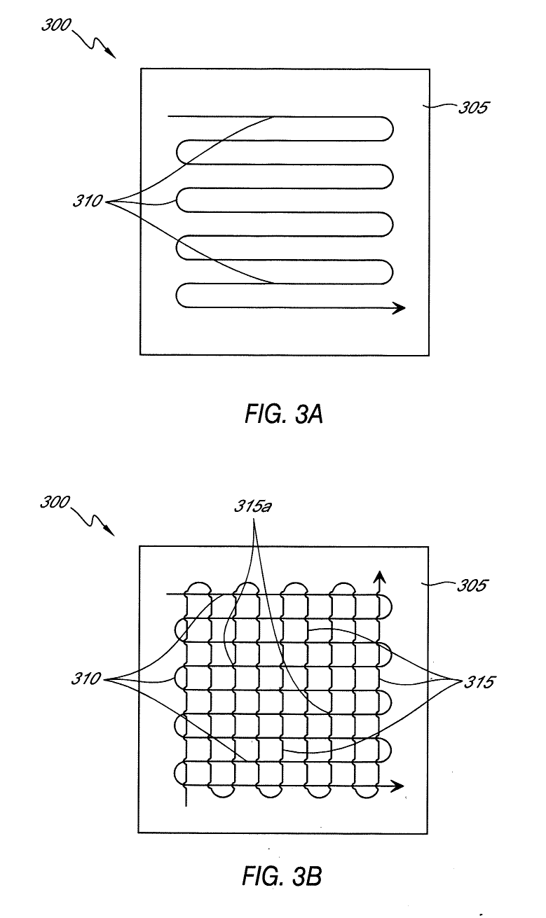

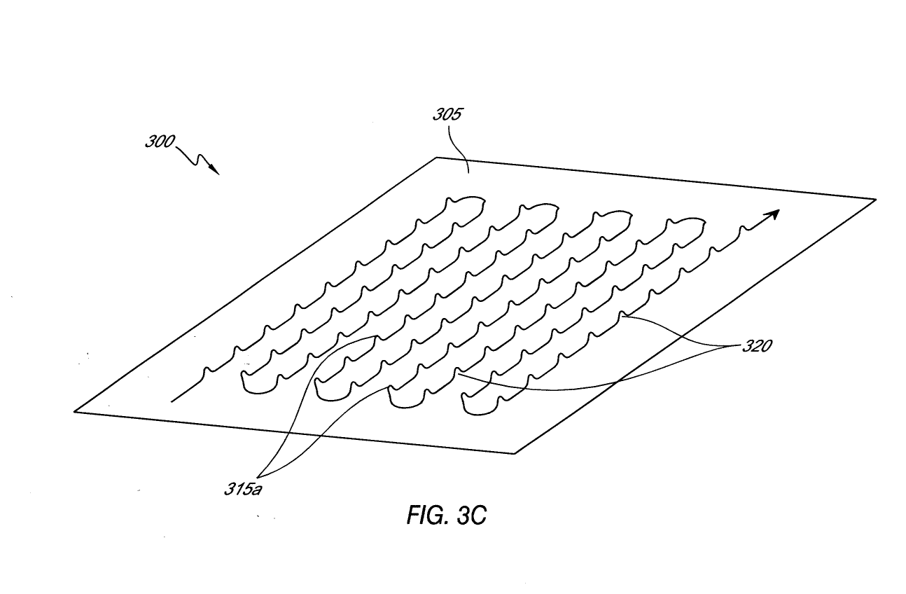

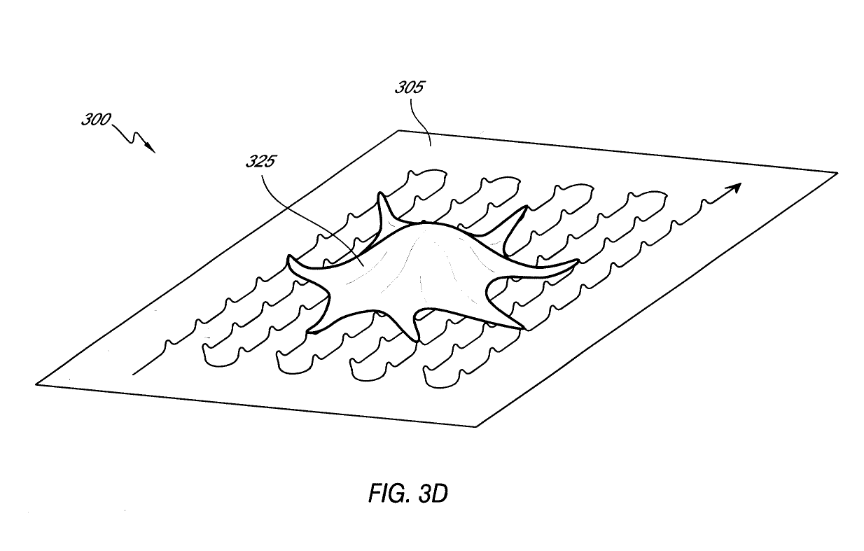

It should be noted that FIGS. 2E (and 3D) is not necessarily drawn to scale. Biological cells can be in the low micron size range (e.g., less than 10 microns). Thus, a single cell may contact hundreds, if not thousands of nanofibers, rather than the handful of fibers or protrusions depicted in FIGS. 2E and 3D.

FIGS. 3A-3D show other illustrative surfaces. The surface 305 can be, for example, a substrate surface or medical device surface. The surface 300 as illustrated in FIGS. 3A-3D can permit improved cell adhesion, for example. As shown in FIG. 3A, one or more first components 310 may be applied to surface 305, for example, to at least partially cover the surface 305. As shown in FIG. 3B, one or more second components 315 may be positioned to at least partially cover the one or more first components 310 and at least part of the substrate 305. The one or more second components 315 may therefore include raised portions 315a, for example, at the intersection of the first and second surface components. As described in further detail below, the one or more first components 310 and/or the one or more second components 315 may be in forms including, but not limited to, strips, lines and/or beading on the substrate. The one or more first surface components (e.g., layers) 310 may include but are not limited to one or more degradable and/or soluble materials. The one or more second surface components (e.g., layers) 315 may include but are not limited to one or more one non-degradable and/or insoluble materials. The materials 310 and/or 315 can be for example, polymers. At least part of the one or more first surface components 310 may be removed (e.g., by application of a solvent or other degrading process). As illustrated in FIG. 3C, the layer of one or more second surface components 315 will at least partially or completely remain on the substrate after partial or complete removal of the layer 310. Thus, cavities 320 may be formed under the raised portions 315a of the one or more second components 315, as shown in FIG. 3C. As illustrated in FIG. 3D, one or more cells 325 may adhere to the surface of the substrate 305. Additional details of illustrative processes for making the surfaces illustrated in FIGS. 3A-3D are provided below.

### Method of Making

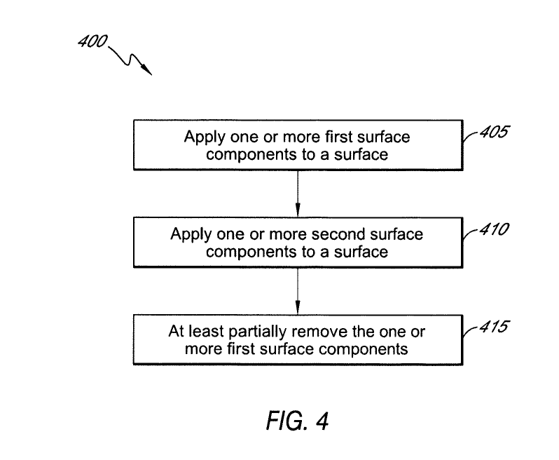

FIG. 4 shows an illustrative process 400 of making a device and/or surface that includes one or more cell-adhesion-promoting surfaces. At step 405, one or more first surface components are applied to or deposited on a surface (e.g., of a device or substrate). Applying a component or agent to a surface may indicate that the component or agent was applied such that it is associated with, supported by, and/or attached to the surface, for example. Thus, in some conditions, at least part of the component or agent can be directly on the surface following application, while in others it is not directly on the surface following application.

Any suitable process can be utilized to apply the first component material. For example, the components can be applied by nano-imprinting, self assembly techniques, phase separation techniques, electrospinning or the like. Nano-imprinting can be utilized, which may not require the use of an imprinting mold. “Self assembly” of nanoparticles or fibers can refer to the process of spontaneous organization into a stable structure without covalent bonds. For example, peptide-amphiphiles can be utilized (hydrophobic tails, cysteine residues-disulfide bonds, etc.). “Phase separation” can refer to the thermodynamic separation of polymer solutions into polymer rich/poor layers, similar to setting a gel. “Electrospinning” refers to processes where an electric field is used to draw a polymer stream out of a solution.

Again, any technique can be utilized which permits the application of the first component onto the substrate or surface. Thus, any process for at least partially coating, brushing, spraying, wiping, dipping, extruding, placing, positioning, depositing and/or injecting the first material can be used.

At step 410, one or more second surface components can be applied to the surface. Although not shown in FIG. 4, it should be noted that one or more additional agents may also be applied to the surface. The second components can be applied using any suitable process, including those described above in connection with step 405.

In some embodiments, the one or more first and second surface components can be positioned (e.g., when the first and second surface components include strips or fibers). The one or more second surface components (e.g., strips) may partly overlap or intersect with the one or more first surface components (e.g., strips), for example, as shown in FIG. 3B. In some instances, a strip of the one or more second surface components overlaps a strip or fiber of the one or more first surface components. For example strip of the second surface component can overlap the strip of the first surface component at a spacing or distance of about 1 to about 1000 nanometers. In some aspects the spacing can be about, at least about, or less than about 5, 10, 20, 50, 70, 100, 150, 200, 250, 300, 350, 400, 500 or 1000 nm, for example, or from at least about 10, 20, 50, 70, 100, 150, 200, 250, 300, 350, 400, or 500 nm to less than about 50, 70, 100, 150, 200, 250, 300, 350, 400, 500 or 1000 nm as appropriate. The components may be positioned to be substantially perpendicular to each other.

The one or more first and second surface components may be configured and/or positioned such that a plurality of cavities are formed beneath the one or more second surface components upon degradation or dissolution of the one or more first surface components. For example, the one or more first surface components may selectively support or raise portions of the one or more second surface components. The one or more one first and second surface components may be configured such that cavities formed upon degradation or dissolution of the one or more first surface components are separated by at least about, about, or less than about 5, 10, 20, 50, 70, 100, 150, 200, 250, 300, 350, 400, 500 or 1000 nm, for example, or from at least about 5, 10, 20, 50, 70, 100, 150, 200, 250, 300, 350, 400, or 500 nm to less than about 20, 50, 70, 100, 150, 200, 250, 300, 350, 400, 500 or 1000 nm as appropriate.

In some instances, amounts of the one or more first surface components and/or an amount of the one or more second surface components that are applied can at least partly determine a spacing of polymeric bumps or raised portions. For example, FIG. 1A shows an example in which a larger amount of the one or more second components 115 is present relative to an amount of nanoparticles 110, as compared to the structure illustrated in FIG. 2A. Thus, the nanoparticles in FIG. 2A contact each other, while those of FIG. 1A do not. The features 115a formed following removal of the nanoparticles 110 are therefore further apart from each other for the example in FIG. 1B than that in FIG. 2B. Other factors, such as a concentration of the one or more first components (e.g., nanoparticles) may also affect the spacing. An amount of the one or more second surface components that is applied also can affect the size of the features (e.g., size of the raised features). For example, applying more of the one or more second surface components may result in larger features. In some embodiments, a size of the one or more first surface components at least partly can determine a size of the raised portions. For example, wider fibers or strips of the one or more first surface components may increase the height of the raised portions.

The one or more first surface components may be applied before the one or more second surface components are applied. The one or more first surface components may be applied after the one or more second surface components are applied. The one or more first surface components and the one or more second surface components may be applied at substantially the same time or at the same time.

At step 415, the one or more first surface components are at least partially removed. In some embodiments, a solvent (e.g., an organic solvent) can be applied to at least part of the surface. Examples of organic solvents include, without being limited thereto, ethyl lactate and ethyl pyruvate, which are non-toxic, photoresist-removing solvents. Other examples of solvents are petroleum-based solvents, such as for example, xylenes, 1,1,1-tricholoroethane, methylene chloride, and N-methylpyrrolidone. These solvents can have some toxicity, and thus, can be washed away or removed prior to use in an application where toxicity is a concern, for example. In some embodiments, heat is applied to at least part of the surface. By controlling, for example, a portion of the surface that has the solvent or heat applied to it, further patterns may be formed on the surface. For example, the surface may contain a first portion to promote cell adhesion and a second portion that does not. In some embodiments, substantially none of the one or more second surface components are removed during step 410, while in others a portion of the one or more second surface components are also removed. For example, in some aspects 0% to 20% of the one or more second surface components are removed, or any value there between. In other aspects 0%-5% or 10% is removed, for example. In one instance, part of the one or more second surface components is positioned above, for example, nanoparticles of the one or more first surface components. Thus, removal of the nanoparticles may effectively detach e.g. a top portion of the one or more second surface components.

Although not shown in FIG. 4, the methods can include methods for making a medical device or methods of making a surface and/or layer that can be used with a medical device. In some instances, the medical device can include a surface to which the first and second surface components have been applied, and from which, at least some of the first surface component has been removed. In other embodiments, the first and second surface components can be applied to a substrate (e.g., a film, piece of a medical device), and the substrate/piece can attached to the medical device. In some instances, cells can be grown on a surface (e.g., of a scaffold or matrix) in vitro and a device that includes the surface is then introduced to the subject.

### First Surface Components

In some embodiments, one or more the first surface components can be utilized with the devices, materials and methods described herein. As mentioned above in the context of FIGS. 1-3, one or more first surface components can be deposited on and/or contacted with a substrate. For example, the one or more first components can include or be composed of a photoresist material, a polypeptide, or any other biodegradable or dissolvable material. The photoresist material can be applied using photomask techniques. For example, photomask techniques can be used to deposit or pattern nanospheres or other nanoparticles onto a substrate. Likewise, photomask techniques can be used to lay down a string or fiber pattern on a substrate. Polypeptides can be deposited, and their amide/carboxy bonds degraded in order to remove the polypeptides. Other types of biopolymers with other bonds can also be applied/deposited and then later degraded.

The one or more first surface components can be removed from the substrate to leave a surface that includes features, for example, features which can permit improved cell adherence to the substrate. As described more fully below, the first surface component(s) can be removed, for example, by a degradation process (biodegradation), by dissolving with a solvent (e.g., an organic solvent or water), or by any other process that will remove the first component. In some aspects a non-toxic or less toxic solvent may be used. The use of such solvents can be desirable for applications where the devices or substrates will be used in vivo to prevent harm to the recipient. For example, in some aspects a non-carcinogenic solvent can be used to remove the photoresist. Examples of solvents include ethyl lactate and ethyl pyruvate, which are non-toxic, photoresist-removing solvents. Other solvents, including some that may be toxic can be used. For example, the solvent may be xylenes, 1,1,1-tricholoroethane, methylene chloride, and N-methylpyrrolidone. In some aspects where toxic solvents are used, the methods and materials can be further treated to remove the toxic solvent from the devices. The first component(s) can be removed preferentially compared to the second component material, for example, more rapidly or to the exclusion of the second component material.

In some aspects the first surface component can include one or more nanoparticles. The first surface component may include substantially homogenous nanoparticles (e.g., in size, shape and/or materials) or non-homogenous nanoparticles. In some aspects, “substantially” homogenous can mean that 30%-100% of the particles have the same size, shape and/or are made of the same material, for example. In further aspects, 60%-99% have the same size, shape and/or material, for example. In still further aspects, 70%, 80%, 90%, 95%, 96%, 97%, and/or 98% can have the same size, shape and/or material. The nanoparticles may include but are not limited to, for example, nanospheres, nanorods, nanofibers, or nanowires. The dimensions of the nanoparticles can be of any suitable size for the particular application and/or for the size of the surface of the device or the layer, for example. In some instances, one or more dimensions (e.g., length, width, diameter, etc.) of the nanoparticles can be between about 1 nm and 1000 nm. In some instances, one or more dimensions of the nanoparticles can be less than about, or about 1000, 500, 300, 100, 50, 30, 10, 5, 3, or 1 nm. In some instances, all dimensions of the nanoparticles can be between about 1 nm and 1000 nm. In some instances, all dimensions of the nanoparticles can be less than about or about 1000, 500, 300, 100, 50, 30, 10, 5, 3, or 1 nm, for example.

A nanoparticle may have an aspect ratio, for example, of less than about 4:1, 3:1, 2:1, 1.5:1, 1.25:1 or 1:1. As one non-limiting example, a nanoparticle in the shape of nanosphere may have an aspect ratio less than about 4:1, 3:1, 2:1, 1.5:1, 1.25:1 or 1:1. The aspect ratio can refer to the ratio of the shortest axis of the nanoparticle to the longest axis of the nanoparticle, where the axes are not necessarily perpendicular. The term “longest dimension” of a nanoparticle can mean, for example, the longest direct path of the nanoparticle. The term “direct path” means, for example, the shortest path contained within the nanoparticle between two points on the surface of the nanoparticle. For example, a helical nanoparticle would have a longest dimension corresponding to the length of the helix if it were stretched out into a straight line.

In some instances, nanoparticles of the one or more first components can be suspended or dispersed within a solution that includes a solvent (e.g., acetone). In some instances, the solution can be partially, substantially or completely saturated with the nanoparticles. For example, partial saturation can mean, for example, that the solution is less than 70% saturated, including for example, between about 5% and 69.9% saturated or between about 10% and about 40% saturated. Substantially saturated can mean, for example, that the solution is about 70%-99.9% saturated or any particular value there between (e.g., 80%, 85%, 90%, 95%, 96%, 97%, 98% or 99%). The solution may then be applied to a surface (e.g., of a medical device or substrate) and the solvent may be removed (e.g., by evaporation). Thus, in some embodiments, the first components can include the solvent, while in other embodiments the first components may include substantially none of the solvent (e.g., after solvent removal).

In some embodiments, the one or more first surface components may include but are not limited to a strip, line, beading or fiber that can, for example, be positioned on a surface or substrate. In some embodiments, the one or more first surface components can be flexible, while in others, it is not. The dimensions ((e.g., height, width, diameter, length, etc.) of the surface component can be of any suitable size for the particular application and/or for the size of the surface of the device or the layer, for example. In some instances, one or more dimensions (e.g., height, width, diameter, length, etc.) of the strip or fiber (or first component of any other configuration or geometry) can be between about 1 nm and 1000 nm. In some instances, one or more dimensions of the strip or fiber can be less than about, or about 1000, 500, 300, 100, 50, 30, 10, 5, 3, or 1 nm, for example.

One, more or all materials (e.g., nanoparticles or strips) of the one or more first components may be degradable or biodegradable. The term “biodegradable,” as used herein, refers to the ability to readily decompose by biological means, such as in water or other biological media or environments, including in vivo and in vitro environments.

One, more or all materials (e.g., nanoparticles or strips) of the one or more first components may be soluble within a solvent. The solvent may include, for example, an organic solvent or an inorganic solvent. Thus, the first components may be organic soluble, meaning that the components are soluble within an organic solvent. One, more or all materials (e.g., nanoparticles) of the one or more first components may be hydrophilic and/or hydrophobic. In some embodiments, one, more or all materials (e.g., nanoparticles or strips) of the one or more first components can include a polymer.

### Second Polymer Surface Components

The one or more second surface components may include one or more polymers. In some aspects the one or more second surface components can be biocompatible. The one or more polymers may include but are not limited to, for example, a polyethylene glycol-based polymer, polyethylene glycol, poly(lactic-co-glycolic-acid), and/or poly(ester amide). The one or more polymers may be characterized by a high molecular weight. The one or more polymers may be characterized by a molecular weight of, for example, at least about, no more than about, or about 0.5, 1, 3, 5, 10, 13, 15, 20, 40, 50, 80, 100, or 500 kg/mol.

The one or more polymers may include, for example, aliphatic polyesters; poly(glycolic acid) and/or copolymers thereof (e.g., poly(glycolide trimethylene carbonate); poly(caprolactone glycolide)); poly(lactic acid) and/or isomers thereof (e.g., poly-L(lactic acid) and/or poly-D Lactic acid) and/or copolymers thereof (e.g. DL-PLA), with and without additives (e.g. calcium phosphate glass), and/or other copolymers (e.g., poly(caprolactone lactide), poly(lactide glycolide), poly(lactic acid ethylene glycol)); poly(ethylene glycol); poly(ethylene glycol) diacrylate; poly(lactide); polyalkylene succinate; polybutylene diglycolate; polyhydroxybutyrate (PHB); polyhydroxyvalerate (PHV); polyhydroxybutyrate/polyhydroxyvalerate copolymer (PHB/PHV); poly(hydroxybutyrate-covalerate); polyhydroxyalkaoates (PHA); polycaprolactone; poly(caprolactone-polyethylene glycol) copolymer; poly(valerolactone); polyanhydrides; poly(orthoesters) and/or blends with polyanhydrides; poly(anhydride-co-imide); polycarbonates (aliphatic); poly(hydroxyl-esters); polydioxanone; polyanhydrides; polyanhydride esters; polycyanoacrylates; poly(alkyl 2-cyanoacrylates); poly(amino acids); poly(phosphazenes); poly(propylene fumarate); poly(propylene fumarate-co-ethylene glycol); poly(fumarate anhydrides); fibrinogen; fibrin; gelatin; cellulose and/or cellulose derivatives and/or cellulosic polymers (e.g., cellulose acetate, cellulose acetate butyrate, cellulose butyrate, cellulose ethers, cellulose nitrate, cellulose propionate, cellophane); chitosan and/or chitosan derivatives (e.g., chitosan NOCC, chitosan NOOC-G); alginate; polysaccharides; starch; amylase; collagen; polycarboxylic acids; poly(ethylester-co-carboxylate carbonate) (and/or other tyrosine derived polycarbonates); poly(iminocarbonate); poly(BPA-iminocarbonate); poly(trimethylene carbonate); poly(iminocarbonate-amide) copolymers and/or other pseudo-poly(amino acids); poly(ethylene glycol); poly(ethylene oxide); poly(ethylene oxide)/poly(butylene terephthalate) copolymer; poly(epsilon-caprolactone-dimethyltrimethylene carbonate); poly(ester amide); poly(amino acids) and conventional synthetic polymers thereof; poly(alkylene oxalates); poly(alkylcarbonate); poly(adipic anhydride); nylon copolyamides; NO-carboxymethyl chitosan NOCC); carboxymethyl cellulose; copoly(ether-esters) (e.g., PEO/PLA dextrans); polyketals; biodegradable polyethers; biodegradable polyesters; polydihydropyrans; polydepsipeptides; polyarylates (L-tyrosine-derived) and/or free acid polyarylates; polyamides (e.g., Nylon 66, polycaprolactam); poly(propylene fumarate-co-ethylene glycol) (e.g., fumarate anhydrides); hyaluronates; poly-p-dioxanone; polypeptides and proteins; polyphosphoester; polyphosphoester urethane; polysaccharides; pseudo-poly(amino acids); starch; terpolymer; (copolymers of glycolide, lactide, or dimethyltrimethylene carbonate); rayon; rayon triacetate; latex; and/copolymers, blends, and/or composites of above.

In some embodiments, the second surface component is characterized as a solid. The one or more second surface components may include but are not limited to a strip or fiber that can, for example, be positioned on a surface or substrate. In some embodiments, the one or more second surface components is flexible, while in others, it is not. The dimensions (e.g., height, width, diameter, length, etc.) of the second surface component can be of any suitable size for the particular application and/or for the size of the surface of the device or the layer, for example. In some instances, one or more dimensions (e.g., height, width, diameter, etc.) of the strip or fiber (or second component of any other configuration or geometry) can be between about 1 nm and 1000 nm, for example. In some instances, one or more dimensions of the strip or fiber can be less than about, or about 1000, 500, 300, 100, 50, 30, 10, 5, 3, or 1 nm, for example.

One, more or all materials (e.g., strips or at least one polymer) of the one or more second components may be substantially non-degradable or substantially non-biodegradable. For example, in some aspects the materials or components can degrade or biodegrade less by less than 20%, including for example less than about 10%, 5%, 4%, 3%, 2%, or 1%. In some instances, it is substantially non-degradable with respect to particular substances, such as water.

One, more or all materials (e.g., strips or at least one polymer) of the one or more second components may be substantially insoluble within a solvent. The solvent may include, for example, an organic solvent or an inorganic solvent. Thus, the second components may be organic insoluble, meaning that the components are substantially insoluble within an organic solvent. One, more or all materials (e.g., strips or at least one polymer) of the one or more second components may be hydrophilic and/or hydrophobic.

In some instances, one, more or all materials (e.g., strips or at least one polymer) of the one or more second components are insoluble within a particular solvent, while one, more or all materials (e.g., strips or nanoparticles) of the one or more first components are soluble within the particular solvent. In some instances, one, more or all materials (e.g., strips or at least one polymer) of the one or more second components are substantially degradable by a particular material, while one, more or all materials (e.g., strips or nanoparticles) of the one or more first components are substantially non-degradable by a particular material.

### Other Agents

In some embodiments, one or more additional agents are provided on a device's surface, with the one or more first and second surface components, and/or with the second surface components. In some embodiments, the one or more first surface components can include the additional agent/s. In some embodiments, the one or more second surface components can include the additional agent/s.

The additional agent may include but is not limited to, for example, a therapeutic agent or biologically active agent. The additional agent may include, for example, an antimicrobial agent, antibacterial agent, anti-fungal agent, anti-viral agent, anti-tumor agent, immunogenic agent, lipid, lipopolysaccharide, hormone and/or growth factor. The additional agent may include heparin, hirudin, hirulog, argatroban, D-phenylalanyl-L-poly-L-arginyl chloromethyl ketone, urokinase, streptokinase, aspirin, ticlopidine, colchicine, cytochalasin, methotrexate, dexamethasone, cyclosporin, trapidal, angiopeptin, dopamine, bromocriptine mesylate, pergolide mesylate, silver, captopril, enalapril, ascorbic acid, alphatocopherol, superoxide dismutase, deferoxyamine, a 21-aminosteroid (lasaroid), a thrombin inhibitor, an antithrombogenic agent, a tissue plasminogen activator, a thrombolytic agent, a fibrinolytic agent, a vasospasm inhibitor, a calcium channel blocker, a nitrate, a nitric oxide promoter, a vasodilator, an antimicrobial agent, an antibiotic agent, a glycoprotein IIb/IIIa inhibitor, an inhibitor of surface glycoprotein receptors, an antiplatelet agent, an antimitotic agent, a microtubule inhibitor agent, a retinoid, an antisecretory agent, an actin inhibitor, a remodeling inhibitor, a deoxyribonucleic acid, an antisense nucleotide, an antiproliferative agent, an anti-cancer chemotherapeutic agent, an anti-inflammatory steroid, a non-steroidal anti-inflammatory agent, an immunosuppressive agent, a PDGF antagonist agent, a growth hormone antagonist agent, an anti-growth factor antibody, a growth factor, a growth factor antagonist, a dopamine agonist agent, a radiotherapeutic agent, a radiopaque agent, a peptide, a protein, an enzyme, an extracellular matrix component, a second angiotensin converting enzyme (ACE) inhibitor, a free radical scavenger agent, an iron chelator agent, an antioxidant agent, and/or a radio labeled agent.

The additional agent may include a cell-adhesion promoter, in that the agent can have physical (e.g.,“sticky” materials) and/or chemical properties that allow, facilitate, and/or enhance cell adherence to the agent while maintaining the integrity of the cell. Cell adhesion may be achieved by specific or non-specific interactions. The cell-adhesion promoter may include but is not limited to, for example, a collagen, gelatin, glucosaminoglycans, fibronectins, lectins, polycations (such polylysine, chitosan and the like), or any other natural or synthetic biological cell adhesion agent.

### Surface Layers

In some embodiments, a surface layer may include one or more surfaces that include the one or more first surface components and the one or more second surface components. A surface layer may include a substrate that includes the one or more second surface components. The surface layer may be configured for attachment to a surface of a device (e.g., a medical device or implantable medical device). In some embodiments, the layer attachment to the surface can include adhering to the surface. The layer may include an adhesive component.

In some embodiments, the layer can include a dissolvable or degradable substrate. Thus, the layer may be applied to the one or more surfaces and the substrate may then be dissolved or degraded, thereby leaving at least part of the one or more second surface components (and—in some embodiments—the one or more first surface components) on, over or attached to the one or more surfaces. In some embodiments, a substrate of the layer is configured to melt following application of heat.

The layer may include a film. The layer may be flexible.

### Medical Devices and Methods of Using

A device (e.g., a medical device) may include one or more surfaces that include the one or more first surface components and the one or more second surface components. A device (e.g., a medical device) may include a surface that includes the one or more second surface components.

Medical devices may include implantable medical devices. The medical device may include, for example, a catheter, fiber, non-woven fabric, vascular graft, porous metal for acetabulum revision, cardiac pacemaker, tachyarrhythmia control device, dental filling material, material used in osteo-synthesis, cardiac patch, suture, soft and hard tissue scaffold and filler, stent, bone void filler intended for the repair of a bone defect, intrauterine device, root canal filler, drug delivery pump, implantable infusion pump, spacer device, implant containing medicinal products, non-medicinal containing implant, and/or scaffold for tissue engineering. The medical device may be one used to promote tissue generation, tissue regeneration or tissue repair. In some embodiments, the medical device includes but is not limited to, for example, a scaffold, matrix or substrate used to promote cell growth in vitro and to be implanted following the cell growth.

The medical device may be one that will be implanted in a specific location. In some instances, it is desirable that cells adhere to the device, thereby securing its position. The adhesion may, for example, reduce the probability that a subject's body rejects the device.

The medical device may include one or more hydrophobic, hydrophilic or amphiphilic surfaces. The medical device may include one or more plasma-treated surfaces or pre-treated surfaces (e.g., to remove an oxide layer).

In some aspects a medical device can be implanted into a subject. In some instances, the medical device can include a surface to which the first and second surface components were applied. In other embodiments, the first and second surface components can be applied to a substrate (e.g., a film), and the substrate can be attached to the medical device. In some instances, cells can be grown on a surface (e.g., on a scaffold or matrix) in vitro and a device that includes the surface then can be introduced to the subject. For example, the surface can be contacted with a suitable cell media and with the desired cells, under conditions conducive to growth for the particular cell(s). Any suitable procedure can be used to grow or attach cells to the surface. Some examples of such procedures and/or references to procedures are found in Ikada, K., “Challenges in tissue engineering,” J. R. Soc. Interface (2006) 3, 589-601; Mendelson K., et al, “Heart Valve Tissue Engineering: Concepts, Approaches, Progress, and Challenges,” Annals of Biomedical Engineering, (2006) 34: 1799-1819; Khademhosseini A., et al., “Microscale technologies for tissue engineering and biology,” PNAS (2006) 103: 2480-2487, each of which is incorporated herein by reference in its entirety, including for all the procedures described and/or referred therein.

The subject may be suffering from, or may be at risk of suffering from, for example, stenosis, a heart condition, a dental condition, a vascular condition, cancer, or an incontinence. The subject may have a heart, coronary vessel or artery, blood vessel, spinal cord, bone, cartilage, tendon, ligament, breast, liver, gallbladder, bile duct, pancreas, intestinal, urinary system tissues, skin, and/or dental tissue that is damaged and/or needing of regeneration. The subject may be undergoing or have recently undergone surgery. Thus, cell adhesion promoted by an implanted medical device may help to seal tissues and/or reduce or prevent post-operation leakage. In some embodiments, the subject needs or desires reconstructive surgery or plastic surgery. The subject may include, but is not limited to, mammals, including a human or other non-human mammals, as well as other non-mammal animals.

The foregoing detailed description has set forth various embodiments of the devices and/or processes via the use of block diagrams, flowcharts, and/or examples. Insofar as such block diagrams, flowcharts, and/or examples contain one or more functions and/or operations, it will be understood by those within the art that each function and/or operation within such block diagrams, flowcharts, or examples can be implemented, individually and/or collectively, by a wide range of hardware, software, firmware, or virtually any combination thereof In one embodiment, several portions of the subject matter described herein may be implemented via Application Specific Integrated Circuits (ASICs), Field Programmable Gate Arrays (FPGAs), digital signal processors (DSPs), or other integrated formats. However, those skilled in the art will recognize that some aspects of the embodiments disclosed herein, in whole or in part, can be equivalently implemented in integrated circuits, as one or more computer programs running on one or more computers (e.g., as one or more programs running on one or more computer systems), as one or more programs running on one or more processors (e.g., as one or more programs running on one or more microprocessors), as firmware, or as virtually any combination thereof, and that designing the circuitry and/or writing the code for the software and or firmware would be well within the skill of one of skill in the art in light of this disclosure. In addition, those skilled in the art will appreciate that the mechanisms of the subject matter described herein are capable of being distributed as a program product in a variety of forms, and that an illustrative embodiment of the subject matter described herein applies regardless of the particular type of signal bearing medium used to actually carry out the distribution. Examples of a signal bearing medium include, but are not limited to, the following: a recordable type medium such as a floppy disk, a hard disk drive, a Compact Disc (CD), a Digital Video Disk (DVD), a digital tape, a computer memory, etc.; and a transmission type medium such as a digital and/or an analog communication medium (e.g., a fiber optic cable, a waveguide, a wired communications link, a wireless communication link, etc.).

Those skilled in the art will recognize that it is common within the art to describe devices and/or processes in the fashion set forth herein, and thereafter use engineering practices to integrate such described devices and/or processes into data processing systems. That is, at least a portion of the devices and/or processes described herein can be integrated into a data processing system via a reasonable amount of experimentation. Those having skill in the art will recognize that a typical data processing system generally includes one or more of a system unit housing, a video display device, a memory such as volatile and non-volatile memory, processors such as microprocessors and digital signal processors, computational entities such as operating systems, drivers, graphical user interfaces, and applications programs, one or more interaction devices, such as a touch pad or screen, and/or control systems including feedback loops and control motors (e.g., feedback for sensing position and/or velocity; control motors for moving and/or adjusting components and/or quantities). A typical data processing system may be implemented utilizing any suitable commercially available components, such as those typically found in data computing/communication and/or network computing/communication systems.

The herein described subject matter sometimes illustrates different components contained within, or connected with, different other components. It is to be understood that such depicted architectures are merely exemplary, and that in fact many other architectures can be implemented which achieve the same functionality. In a conceptual sense, any arrangement of components to achieve the same functionality is effectively “associated” such that the desired functionality is achieved. Hence, any two components herein combined to achieve a particular functionality can be seen as “associated with” each other such that the desired functionality is achieved, irrespective of architectures or intermedial components. Likewise, any two components so associated can also be viewed as being “operably connected”, or “operably coupled”, to each other to achieve the desired functionality, and any two components capable of being so associated can also be viewed as being “operably couplable”, to each other to achieve the desired functionality. Specific examples of operably couplable include but are not limited to physically mateable and/or physically interacting components and/or wirelessly interactable and/or wirelessly interacting components and/or logically interacting and/or logically interactable components.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity.

It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should typically be interpreted to mean “at least one” or “one or more”); the same holds true for the use of definite articles used to introduce claim recitations. In addition, even if a specific number of an introduced claim recitation is explicitly recited, those skilled in the art will recognize that such recitation should typically be interpreted to mean at least the recited number (e.g., the bare recitation of “two recitations,” without other modifiers, typically means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

While various aspects and embodiments have been disclosed herein, other aspects and embodiments will be apparent to those skilled in the art. The various aspects and embodiments disclosed herein are for purposes of illustration and are not intended to be limiting, with the true scope and spirit being indicated by the following claims.

What is claimed is:

1. A method of manufacturing an implantable medical device, the method comprising:
at least partially contacting the medical device with one or more first surface components including one or more nanoparticles; and
at least partially contacting the medical device with one or more second surface components including one or more polymers, such that the second surface components at least partly surrounding the first surface components.

2. The method of claim 1, wherein the one or more first surface components are organic soluble or organic insoluble.

3. The method of claim 1, wherein the polymer includes a polyethylene glycol-based polymer.

4. The method of claim 1, wherein the at least partially contacting the medical device with the one or more first surface components includes one or more of at least partially coating the medical device with the one or more first surface components, and brushing, spraying, wiping, dipping, extruding and/or injecting the medical device with the first component.

5. The method of claim 1, wherein the at least partially contacting the medical device with the one or more second surface components includes one or more of at least partially coating the medical device with the one or more second surface components, and brushing, spraying, wiping, dipping, extruding and/or injecting the medical device with the second component.

6. The method of claim 1, further comprising preparing a coating solution including the first and second components, wherein at least partially contacting the medical device with the one or more first surface components and at least partially contacting the medical device with the one or more second surface components includes at least partially contacting the medical device with the coating solution.

7. The method of claim 6, wherein the coating solution further includes a biologically active agent.

8. The method of claim 1, wherein the polymer is characterized by a molecular weight of at least about 10,000 g/mol or about 20,000 g/mol.

9. The method of claim 1, wherein the nanoparticles include a polymer.

10. The method of claim 1, wherein adjacent nanoparticles contact each other.

11. The method of claim 1, wherein the nanoparticles include nanospheres.

12. The method of claim 1, further including contacting at least part of the medical device with an organic solvent.

13. The method of claim 12, wherein the contacting the medical device with an organic solvent detaches at least part of the one or more first surface components from the medical device.

14. A method of manufacturing a layer configured for attachment to a surface of an implantable medical device, the method comprising:
at least partially contacting the medical device with one or more first surface components including one or more nanoparticles; and
at least partially contacting the medical device with one or more second surface components including one or more polymers, such that the second surface components at least partly surrounding the first surface components.

15. The method of claim 14, wherein the layer includes an adhesive component.

16. The method of claim 14, further comprising at least partially removing the one or more first components.

17. A method of manufacturing an implantable medical device, the method comprising:
positioning one or more first components at least partially over one or more surfaces of the medical device, the one or more first components including one or more biodegradable polymers; and
positioning one or more second components at least partially over the one or more first components, the one or more second components including one or more non-biodegradable polymers.

18. The method of claim 17, wherein at least one of the one or more first components or the one or more second components includes a strip or a fiber.

19. The method of claim 17, wherein the one or more first components include at least one strip.

20. The method of claim 19, wherein the first component strips are positioned substantially perpendicular to the second component strips.

21. The method of claim 19, wherein the strips are positioned to form a plurality of intersections between the biodegradable polymer strip and the non-biodegradable polymer strip.

22. The method of claim 19, wherein the strips are positioned such that the first component strip selectively raises portions of the second component strip.

23. The method of claim 17, wherein the one or more second components include at least one strip.

24. The method of claim 17, wherein the one or more second components include a polyethylene glycol-based polymer.

25. The method of claim 17, further comprising removing the one or more first components.

26. The method of claim 25, wherein the removing includes contacting the one or more first components with a solvent.

27. The method of claim 25, wherein the removing results in the formation of a plurality of cavities between the one or more second components and the surface.

28. The method of claim 17, wherein the medical device is selected from the group consisting of catheters, fibers, non-woven fabrics, vascular grafts, porous metals for acetabulum revision, cardiac pacemakers, tachyarrhythmia control devices, dental filling materials, materials used in osteo-synthesis, cardiac patches, sutures, soft and hard tissue scaffolds and fillers, stents, bone void fillers intended for the repair of bone defects, intrauterine devices, root canal fillers, drug delivery pumps, implantable infusion pumps, spacer devices, implants containing medicinal products, and scaffolds for tissue engineering.

29. A method of manufacturing a layer configured for attachment to a surface of an implantable medical device, the method comprising:
positioning a biodegradable polymer strip at least partially over a substrate; and
positioning one or more non-biodegradable polymers at least partially over the biodegradable polymer strip.

30. An implantable medical device comprising:
one or more first components including one or more biodegradable polymers at least partially covering one or more surfaces of the medical device; and
one or more second components including one or more non-biodegradable polymers at least partially positioned over the first surface layer.

31. The device of claim 30, wherein at least one of the one or more first components and the one or more second components include a strip or a fiber.

32. The device of claim 30, wherein the first and second components are configured such that a plurality of cavities is formed between the one or more second components and the surface upon degradation of the one or more first components.

33. The device of claim 30, wherein adjacent cavities are separated by a distance greater than about 50 nm and less than about 300 nm.

34. The device of claim 30, wherein the strips are positioned to form a plurality of intersections between the biodegradable polymer strip and the non-biodegradable polymer strip.

35. An implantable medical device comprising:
one or more first surface components including one or more nanoparticles; and
one or more second surface components including one or more polymers, the second surface components at least partly surrounding the first surface components.

36. The device of claim 30, wherein the nanoparticles include nanospheres.

37. A surface layer comprising:
one or more first surface components including one or more nanoparticles; and
one or more second surface components including one or more polymers, the second surface components at least partly surrounding the first surface components,
wherein the surface layer is configured for attachment to a surface of an implantable medical device.

38. A layer configured for attachment to a surface of an implantable medical device, the layer comprising:
a biodegradable polymer strip over a substrate; and
one or more non-biodegradable polymers over the biodegradable polymer strip.
